(19) United States
(12) Patent Application Publication
(10) Pub. No.: US 2024/0285249 A1
(43) Pub. Date: Aug. 29, 2024

(54) SENSOR HOUSING FOR INTRALUMINAL SENSING DEVICE

(71) Applicants: KONINKLIJKE PHILIPS N.V., EINDHOVEN (NL); PHILIPS IMAGE GUIDED THERAPY CORPORATION, SAN DIEGO, CA (US)

(72) Inventor: Justin Patrick MAY, RANCHO MISSION VIEJO, CA (US)

(21) Appl. No.: 18/574,358

(22) PCT Filed: Jun. 27, 2022
(86) PCT No.: PCT/EP2022/067466

§ 371 Date: Dec. 27, 2023

Related U.S. Application Data
(60) Provisional application No. 63/217,417, filed on Jul. 1, 2021.

(51) Int. Cl. A61B 8/06; A61B 8/12

(52) U.S. Cl. CPC A61B 8/06; A61B 8/12

(57) ABSTRACT

An intraluminal sensing device may include a flexible elongate member, a housing, and a sensor. The flexible elongate member may include a distal portion and a proximal portion and may be configured to be positioned within a body lumen of a patient. The housing may be positioned at the distal portion of the flexible elongate member. The housing may include a conductive material and a non-conductive material. The sensor may include an exposed conductive surface and may be configured to obtain physiological data while positioned within the body lumen. The sensor may be positioned within the housing such that the exposed conductive surface contacts the non-conductive material of the housing and is electrically isolated from the conductive material.

### TECHNICAL FIELD

The present disclosure relates generally to intraluminal sensing devices and, in particular, to intraluminal sensing devices comprising a housing that includes a non-conductive material and a conductive material. For example, a sensor of the intraluminal sensing device may be positioned within the housing such that an exposed conductive surface of the sensor contacts the non-conductive material and is electrically isolated from the conductive material.

### BACKGROUND

Intraluminal sensing devices, such as intravascular sensing devices, may include a sensor configured to obtain physiological data while positioned within a lumen, such as a blood vessel. For instance, such devices may include an imaging apparatus, a flow sensor, or a pressure sensor sized and shaped to be positioned within the lumen and configured to capture images, flow data, or pressure data within the lumen. In some cases, the sensor an intraluminal sensing device may be coated in an insulating material, such as parylene. In particular, the sensor may be coated with the insulating material before the sensor is added to an assembly of the intraluminal sensing device, such as before the sensor is positioned within a housing of the intraluminal sensing device.

Coating the sensor with an insulating material may be a resource-intensive process and may reduce the performance (e.g., the accuracy) of the sensor. For instance, the coating may involve specific equipment (e.g., machinery) and may increase production time and/or costs for the intraluminal sensing device. Further, application of a precise thickness of insulating material may be relatively difficult to achieve (e.g., may have a relatively low reproducibility), which may introduce variability in device performance across intraluminal sensing devices. Moreover, the coating may stiffen a sub-assembly that includes the sensor, which may complicate assembly of the intraluminal sensing device and may affect alignment and/or performance of the sensor in the manufactured device. Additionally, interfaces with and materials applied to the sensor cause distortions in the data captured by sensor.

### SUMMARY

Disclosed herein is an intraluminal sensing device (e.g., an intravascular sensing device) that may be configured to obtain physiological data while positioned within a lumen, such as a blood vessel. The device includes a flexible elongate member (e.g., a guidewire and/or a catheter), a housing, and a sensor (e.g., a sensing component), which may be configured to obtain the physiological data and may be positioned within the housing. The sensor may include an exposed conductive surface (e.g., a surface that lacks an insulating layer or coating) and one or more electrical and/or electronic components, such as an ultrasound transducer. Maintaining an exposed conductive surface on the sensor may reduce the complexity and resources involved with assembling the intraluminal device (e.g., positioning the sensor within the sensor) and may improve performance of the sensor itself. However, electrical power and/or signals may be provided to the electronic components of the sensor to control operation of the sensor and/or to communicate data with the sensor. Accordingly, because the housing may include (e.g., may be formed from) a conductive material, such as a metal, contact between a conductive surface lacking an insulating layer and the housing may short (e.g., cause an electrical failure at) the sensor and/or its electrical components. To prevent such an electrical failure, the housing may also include a non-conductive material, and the sensor may be positioned within the housing such that the exposed conductive surface of the sensor contacts the non-conductive material of the housing and is electrically isolated from the conductive material of the housing. For instance, the housing may include a hollow interior that is defined by a continuous surface, where first portion of the continuous surface may include the conductive material and a second portion of the continuous surface may include the non-conductive material. In such cases, the sensor may be positioned such that the exposed conductive surface contacts the first portion and is electrically isolated from the second portion.

In an exemplary aspect, an intraluminal sensing device may include a flexible elongate member, a housing, and a sensor. The flexible elongate member may include a distal portion and a proximal portion and may be configured to be positioned within a body lumen of a patient. The housing may be positioned at the distal portion of the flexible elongate member. The housing may include a conductive material and a non-conductive material. The sensor may include an exposed conductive surface and may be configured to obtain physiological data while positioned within the body lumen. The sensor may be positioned within the housing such that the exposed conductive surface contacts the non-conductive material of the housing and is electrically isolated from the conductive material.

In some aspects, the exposed conductive surface includes a perimeter of the sensor. In some aspects, the non-conductive material includes a ceramic material. In some aspects, the non-conductive material includes a polymer. In some aspects, the housing includes a hollow interior defined by a continuous surface. A first portion of the continuous surface may include the conductive material. A different, second portion of the continuous surface may include the non-conductive material. In some aspects, the housing includes a plurality of layers formed atop one another such that the plurality of layers defines the continuous surface. A first layer of the plurality of layers may include the conductive material and may be disposed within the first portion of the continuous surface. A different, second layer of the plurality of layers may include the non-conductive material and may be disposed within the second portion of the continuous surface. In some aspects, the sensor is positioned within the hollow interior such that the exposed conductive surface contacts the second portion of the continuous surface and is spaced from the first portion of the continuous surface. In some aspects, the housing includes a first projection extending within a hollow interior of the housing. The first projection may include the non-conductive material and may be in contact with the exposed conductive surface. In some aspects, the first projection surrounds a perimeter of the exposed conductive surface. In some aspects, the housing includes a different, second projection extending within the hollow interior. The second projection may include the non-conductive material and may be in contact with the exposed conductive surface. The first projection may be arranged to contact the exposed conductive surface over a first portion of the exposed conductive surface. The second projection may be arranged to contact the exposed conductive surface over a different, second portion of the exposed conductive surface. The second portion may be spaced from the first portion. In some aspects, the intraluminal sensing device further includes an air gap positioned between the sensor and the housing. The air gap may include an acoustic backing layer. In some aspects, the sensor includes a flow sensor. In some aspects, the intraluminal sensing device further includes a wire assembly electrically coupled to the exposed conductive surface. In some aspects, intraluminal sensing device further includes an adhesive positioned between the sensor and the housing. In some aspects, the housing includes a coating of the non-conductive material.

In an exemplary aspect, an intraluminal flow-sensing device may include a guidewire, a housing, and a flow sensor. The guidewire may include a distal portion and a proximal portion and may be configured to be positioned within a blood vessel of a patient. The housing may be positioned at the distal portion of the guidewire and may include a hollow interior defined by a continuous surface. A first portion of the continuous surface may include a conductive material and a different, second portion of the continuous surface may include a non-conductive material. The flow sensor may include an exposed conductive surface and may be configured to obtain intravascular flow data while positioned within the blood vessel. The flow sensor may be positioned within the hollow interior of the housing such that the exposed conductive surface contacts the second portion and is electrically isolated from the first portion.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic side view of an intravascular sensing system that includes an intravascular device comprising a housing with a conductive material and a non-conductive material, in accordance with at least one embodiment of the present disclosure.

FIG. 2A is a diagrammatic cross-sectional side view of a sensor assembly, in accordance with at least one embodiment of the present disclosure.

FIG. 2B is a diagrammatic, cross-sectional view of cut plane A-A from FIG. 2A, in accordance with at least one embodiment of the present disclosure.

FIG. 3A is a diagrammatic cross-sectional side view of a sensor assembly, in accordance with at least one embodiment of the present disclosure.

FIG. 3B is a diagrammatic, cross-sectional view of cut plane B-B from FIG. 3A, in accordance with at least one embodiment of the present disclosure.

FIG. 4A is a diagrammatic cross-sectional side view of a sensor assembly, in accordance with at least one embodiment of the present disclosure.

FIG. 4B is a diagrammatic, cross-sectional view of cut plane C-C from FIG. 4A, in accordance with at least one embodiment of the present disclosure.

FIG. 5 is a diagrammatic cross-sectional side view of a sensor assembly, in accordance with at least one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. Additionally, while the description below may refer to blood vessels, it will be understood that the present disclosure is not limited to such applications. For example, the devices, systems, and methods described herein may be used in any body chamber or body lumen, including an esophagus, veins, arteries, intestines, ventricles, atria, or any other body lumen and/or chamber. For the sake of brevity, however, the numerous iterations of these combinations will not be described separately.

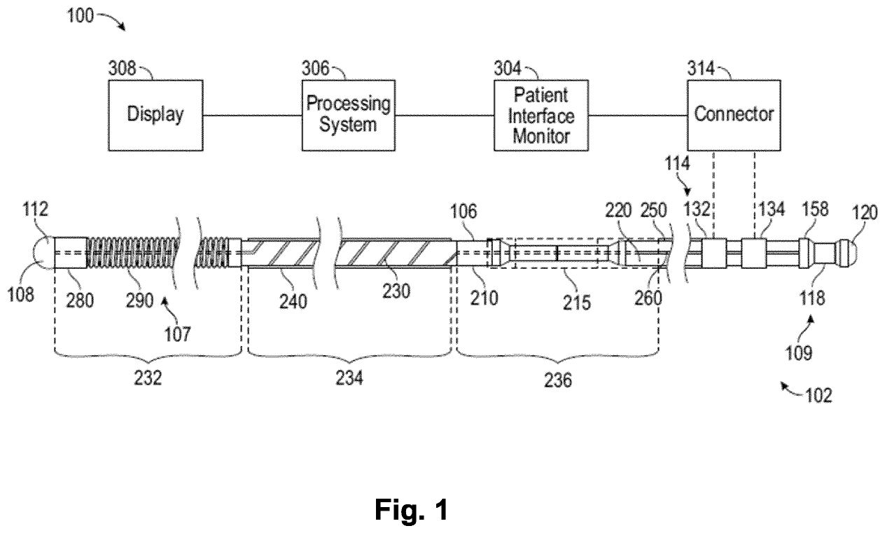

FIG. 1 is a diagrammatic side view of an intravascular sensing system 100 (e.g., an intraluminal sensing system) that includes an intravascular device 102 (e.g., an intraluminal sensing device) comprising a sensing component 112 positioned within a housing 280 that includes a conductive material and a non-conductive material, according to aspects of the present disclosure. The intravascular device 102 can be an intravascular guidewire sized and shaped for positioning within a vessel of a patient. The intravascular device 102 can include a distal tip 108 and a sensing component 112. The sensing component 112 can be an electronic, electromechanical, mechanical, optical, and/or other suitable type of sensor. For example, the sensing component 112 can be a flow sensor configured to measure the velocity of blood flow within a blood vessel of a patient, a pressure sensor configured to measure a pressure of blood flowing within the vessel, or another type of sensor including but not limited to a temperature or imaging sensor. For example, flow data obtained by a flow sensor can be used to calculate physiological variables such as coronary flow reserve (CFR). Pressure data obtained by a pressure sensor may for example be used to calculate a physiological pressure ratio (e.g., FFR, iFR, Pd/Pa, or any other suitable pressure ratio). An imaging sensor may include an intravascular ultrasound (IVUS), intracardiac echocardiography (ICE), optical coherence tomography (OCT), or intravascular photoacoustic (IVPA) imaging sensor.

In some embodiments, the sensing component 112 may include one or more transducers, such as one or more ultrasound transducer elements. The one or more ultrasound transducer element (e.g., an acoustic element) may be configured to emit ultrasound energy and receive echoes corresponding to the emitted ultrasound energy. Further, the one or more ultrasound transducer elements may include a piezoelectric/piezoresistive element, a piezoelectric micromachined ultrasound transducer (PMUT) element, a capacitive micromachined ultrasound transducer (CMUT) element, and/or any other suitable type of ultrasound transducer element. The one or more ultrasound transducer elements may further be in communication with (e.g., electrically coupled to) electronic circuitry. For example, the electronic circuitry can include one or more transducer control logic dies. The electronic circuitry can include one or more integrated circuits (IC), such as application specific integrated circuits (ASICs). In some embodiments, one or more of the ICs can include a microbeamformer (uBF). In other embodiments, one or more of the ICs includes a multiplexer circuit (MUX).

Further the one or more transducers of the sensing component 112 may be arranged in any suitable configuration. For example, an imaging sensor can an array of ultrasound transducer elements, such as a linear array, a planar array, a curved array, a curvilinear array, a circumferential array, an annular array, a phased array, a matrix array, a one-dimensional (1D) array, a 1.x dimensional array (e.g., a 1.5D array), or a two-dimensional (2D) array. The array of transducer elements (e.g., one or more rows, one or more columns, and/or one or more orientations) can be uniformly or independently controlled and activated. The array can be configured to obtain one-dimensional, two-dimensional, and/or three-dimensional images of patient anatomy.

In an exemplary embodiment, the sensing is a flow sensor, which includes a single ultrasound transducer element, such as the transducer elements described above. The transducer element emits ultrasound signals and receives ultrasound echoes reflected from anatomy (e.g., flowing fluid, such as blood). The transducer element generates electrical signals representative of the echoes. The signal-carrying filars carry this electrical signal from the sensor at the distal portion to the connector at the proximal portion. The processing system processes the electrical signals to extract the flow velocity of the fluid. In other embodiments, the device 102 may be used to examine any number of anatomical locations and tissue types, including without limitation, organs including the liver, heart, kidneys, gall bladder, pancreas, lungs: ducts: intestines: nervous system structures including the brain, dural sac, spinal cord and peripheral nerves: the urinary tract: as well as valves within the blood, chambers or other parts of the heart, and/or other systems of the body. In addition to natural structures, the device 102 may be used to examine man-made structures such as, but without limitation, heart valves, stents, shunts, filters and other devices. In some embodiments, the sensing component 112 may include an imaging component (e.g., an intravascular ultrasound imaging component), a measurement component (e.g., a pressure, flow, or temperature sensor) and/or a treatment component (e.g., an ablation component).

In some embodiments the sensing component 112 may be fully or partially enclosed within a housing 280. In some embodiments, the sensing component 112 is located at or near the distal end of a flexible elongate member, and may include a distal tip 108 (e.g., an atraumatic tip). In some embodiments, one or more electronic components, such as the sensing component 112, can be located at the distal portion of the flexible elongate member. For example, the one or more electronic components can be located at the distal tip (a leading edge of the flexible elongate member and/or where the distal portion terminates) or proximally spaced from the distal end (by, e.g., 0.5 cm, 1 cm, 1.5 cm, 2 cm, 3 cm, 4 cm, 5 cm, and/or other suitable values both larger and smaller). Some embodiments of the intravascular device 102 include multiple, different electronic and/or sensing components (e.g., a pressure sensor and a flow sensor, or any other quantity or combination of sensors). In such embodiments, a first electronic component can be positioned at the distal tip of the flexible elongate member and the second electronic component can be spaced from the distal tip and/or from the first electronic component (by, e.g., 0.5 cm, 1 cm, 1.5 cm, 2 cm, 3 cm, 4 cm. 5 cm, and/or other suitable values both larger and smaller). In some embodiments, power, control signals, and electrical ground or signal return may be provided by the multi-filar conductor bundle 230, which includes multiple conductive filars. The conductive filars may, for example, be made of pure copper, or of a copper alloy such as BeCu or AgCu.

The intravascular device 102 includes a flexible elongate member 106. The sensing component 112 is disposed at the distal portion 107 of the flexible elongate member 106. The sensing component 112 can be mounted at the distal portion 107 within a housing 280 in some embodiments. A flexible tip coil 290 extends proximally from the housing 280 at the distal portion 107 of the flexible elongate member 106. A connection portion 114 located at a proximal end of the flexible elongate member 106 includes conductive portions 132, 134. In some embodiments, the conductive portions 132, 134 can be conductive ink that is printed and/or deposited around the connection portion 114 of the flexible elongate member 106. In some embodiments, the conductive portions 132, 134 are conductive, metallic rings that are positioned around the flexible elongate member. A locking section is formed by collar 118 and knob 120 are disposed at the proximal portion 109 of the flexible elongate member 106.

The intravascular device 102 in FIG. 1 includes a distal core wire 210 and a proximal core wire 220. The distal core 210 and the proximal core 220 are metallic components forming part of the body of the intravascular device 102. For example, the distal core 210 and the proximal core 220 are flexible metallic rods that provide structure for the flexible elongate member 106. The diameter of the distal core 210 and the proximal core 220 can vary along its length. A joint between the distal core 210 and proximal core 220 is surrounded and contained by a hypotube 215.

In some embodiments, the intravascular device 102 comprises a distal assembly and a proximal assembly that are electrically and mechanically joined together, which provides for electrical communication between the sensing component 112 and the conductive portions 132, 134. For example, flow data obtained by the sensing component 112 (in this example, sensing component 112 is a flow sensor) can be transmitted to the conductive portions 132, 134. Control signals (e.g., operating voltage, start/stop commands, etc.) from a processor system 306 in communication with the intravascular device 102 can be transmitted to the sensing component 112 via a connector 314 that is attached to the conductive portions 132, 134. The distal subassembly can include the distal core 210. The distal subassembly can also include the sensing component 112, a multi-filar conductor bundle 230, and/or one or more layers of insulative polymer/plastic 240 surrounding the conductive members 230 and the core 210. For example, the polymer/plastic layer(s) can insulate and protect the conductive members of the multi-filar cable or conductor bundle 230. The proximal subassembly can include the proximal core 220. The proximal subassembly can also include one or more layers of polymer layer(s) 250 (hereinafter polymer layer 250) surrounding the proximal core 220 and/or conductive ribbons 260 embedded within the one or more insulative and/or protective polymer layer(s) 250. In some embodiments, the proximal subassembly and the distal subassembly can be separately manufactured. During the assembly process for the intravascular device 102, the proximal subassembly and the distal subassembly can be electrically and mechanically joined together. As used herein, flexible elongate member can refer to one or more components along the entire length of the intravascular device 102, one or more components of the proximal subassembly (e.g., including the proximal core 220, etc.), and/or one or more components the distal subassembly 210 (e.g., including the distal core 210, etc.). The joint between the proximal core 220 and distal core 210 is surrounded by the hypotube 215.

In various embodiments, the intravascular device 102 can include one, two, three, or more core wires extending along its length. For example, in one embodiment, a single core wire extends substantially along the entire length of the flexible elongate member 106. In such embodiments, a locking section 118 and a section 120 can be integrally formed at the proximal portion of the single core wire. The sensing component 112 can be secured at the distal portion of the single core wire. In other embodiments, such as the embodiment illustrated in FIG. 1, the locking section 118 and the section 120 can be integrally formed at the proximal portion of the proximal core 220. The sensing component 112 can be secured at the distal portion of the distal core 210. The intravascular device 102 includes one or more conductive members in a multi-filar conductor bundle 230 (e.g., a wire assembly) in communication with the sensing component 112. For example, the conductor bundle 230 can include one or more electrical wires that are directly in communication with the sensing component 112. In some instances, the conductive members 230 are electrically and mechanically coupled to the sensing component 112 by, e.g., soldering. In some instances, the conductor bundle 230 comprises two or three electrical wires (e.g., a bifilar cable or a trifilar cable). An individual electrical wire can include a bare metallic conductor, or a metallic conductor surrounded by one or more insulating layers. The multi-filar conductor bundle 230 can extend along a length of the distal core 210. For example, at least a portion of the conductive members 230 can be helically, or spirally, wrapped around an entire length of the distal core 210, or a portion of the length of the distal core 210.

The intravascular device 102 includes one or more conductive ribbons 260 at the proximal portion of the flexible elongate member 106. The conductive ribbons 260 are embedded within polymer layer(s) 250. The conductive ribbons 260 are directly in communication with the conductive portions 132 and/or 134. In some instances, the multi-filar conductor bundle 230 is electrically and mechanically coupled to the sensing component 112 by, e.g., soldering. In some instances, the conductive portions 132 and/or 134 comprise conductive ink (e.g., metallic nano-ink, such as silver or gold nano-ink) that is deposited or printed directed over the conductive ribbons 260.

As described herein, electrical communication between the conductive members 230 and the conductive ribbons 260 can be established at the connection portion 114 of the flexible elongate member 106. By establishing electrical communication between the conductor bundle 230) and the conductive ribbons 260, the conductive portions 132, 134 can be in electrically communication with the sensing component 112.

In some embodiments represented by FIG. 1, intravascular device 102 includes a locking section 118 and a section 120. To form locking section 118, a machining process is necessary to remove polymer layer 250 and conductive ribbons 260 in locking section 118 and to shape proximal core 220 in locking section 118 to the desired shape. As shown in FIG. 1, locking section 118 includes a reduced diameter while section 120 has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons.

In some embodiments, a connector 314 provides electrical connectivity between the conductive portions 132, 134 and a patient interface module or monitor 304. The patient interface module (PIM) 304 may in some cases connect to a console or processing system 306, which includes or is in communication with a display 308. In some embodiments, the patient interface module 304 includes signal processing circuitry, such as an analog-to-digital converter (ADC), analog and/or digital filters, signal conditioning circuitry, and any other suitable signal processing circuitry for processing the signals provided by the sensing component 112 for use by the processing system 306.

The system 100 may be deployed in a catheterization laboratory having a control room. The processing system 306 may be located in the control room. Optionally, the processing system 306 may be located elsewhere, such as in the catheterization laboratory itself. The catheterization laboratory may include a sterile field while its associated control room may or may not be sterile depending on the procedure to be performed and/or on the health care facility. In some embodiments, device 102 may be controlled from a remote location such as the control room, such than an operator is not required to be in close proximity to the patient.

The intraluminal device 102, PIM 304, and display 308 may be communicatively coupled directly or indirectly to the processing system 306. These elements may be communicatively coupled to the medical processing system 306 via a wired connection such as a standard copper multi-filar conductor bundle 230. The processing system 306 may be communicatively coupled to one or more data networks, e.g., a TCP/IP-based local area network (LAN). In other embodiments, different protocols may be utilized such as Synchronous Optical Networking (SONET). In some cases, the processing system 306 may be communicatively coupled to a wide area network (WAN).

The PIM 304 transfers the received signals to the processing system 306 where the information is processed and displayed on the display 308. The console or processing system 306 can include a processor and a memory. The processing system 306 may be operable to facilitate the features of the intravascular sensing system 100 described herein. For example, the processor can execute computer readable instructions stored on the non-transitory tangible computer readable medium.

The PIM 304 facilitates communication of signals between the processing system 306 and the intraluminal device 102. In some embodiments, the PIM 304 performs preliminary processing of data prior to relaying the data to the processing system 306. In examples of such embodiments, the PIM 304 performs amplification, filtering, and/or aggregating of the data. In an embodiment, the PIM 304 also supplies high- and low-voltage DC power to support operation of the intraluminal device 102 via the multi-filar conductor bundle 230.

The multi-filar cable or transmission line bundle 230 can include a plurality of conductors, including one, two, three, four, five, six, seven, or more conductors. The multi-filar conductor bundle 230 can be positioned along the exterior of the distal core 210. The multi-filar conductor bundle 230 and the distal core 210 can be overcoated with an insulative and/or protective polymer 240. In the example shown in FIG. 1, the multi-filar conductor bundle 230 includes two straight portions 232 and 236, where the multi-filar conductor bundle 230 extends linearly and parallel to a longitudinal axis of the flexible elongate member 106 on the exterior of the distal core 210, and a helical or spiral portion 234, where the multi-filar conductor bundle 230 is wrapped around the exterior of the distal core 210. In some embodiments, the multi-filar conductor bundle 230 only includes a straight portion or only includes a helical or spiral portion. In general, the multi-filar conductor bundle 230 can extend in a linear, wrapped, non-linear, or non-wrapped manner, or any combination thereof. Communication, if any, along the multi-filar conductor bundle 230 may be through numerous methods or protocols, including serial, parallel, and otherwise, wherein one or more filars of the bundle 230 carry signals. One or more filars of the multi-filar conductor bundle 230 may also carry direct current (DC) power, alternating current (AC) power, or serve as an electrical ground connection.

The display or monitor 308 may be a display device such as a computer monitor, a touch-screen display, a television screen, or any other suitable type of display. The monitor 308 may be used to display selectable prompts, instructions, and visualizations of imaging data to a user. In some embodiments, the monitor 308 may be used to provide a procedure-specific workflow to a user to complete an intraluminal imaging procedure.

Before continuing, it should be noted that the examples described above are provided for purposes of illustration, and are not intended to be limiting. Other devices and/or device configurations may be utilized to carry out the operations described herein.

In some embodiments, the sensing component 112 includes a conductive surface. In some cases, the housing 280 also includes a conductive surface or portion. For instance, the housing 280 may be constructed from a metal. Contact between the conductive surface of the sensing component 112 and the conductive portion of the housing may thus short (e.g., cause an electrical failure at) the sensing component 112 and/or the electronics coupled to the sensing component. Accordingly, in some embodiments, the sensing component 112 may be coated in an insulating material, such as parylene, to prevent the electrical short between the sensor and the housing. By coating the sensing component 112 in parylene, for example, the sensing component 112 may be electrically isolated from the housing. However, coating the sensing component 112 in parylene or another insulating material may be a resource-intensive process. For instance, the coating may involve specific equipment (e.g., machinery) and may increase production time and/or costs for the intravascular device 102. Further, application of a precise coating may be relatively difficult to achieve (e.g., may have a relatively low reproducibility), which may introduce variability in performance between different intravascular devices. Moreover, the coating may stiffen an assembly including the sensing component 112, which may complicate assembly of the intravascular device 102. In particular, positioning the assembly within the housing 280 may be more difficult with a stiffer assembly. As a result, the assembly may be misaligned within the housing 280, which may affect a performance of the sensing component 112. Additionally, interfaces with and materials applied to the sensing component 112 that have different acoustic impedances from one another and/or from the sensing component 112 may distort data captured by the sensing component 112 (e.g., may cause impedance mismatches). Accordingly, reducing or eliminating coatings or other materials applied to the sensing component 112 may improve the performance of the sensing component 112.

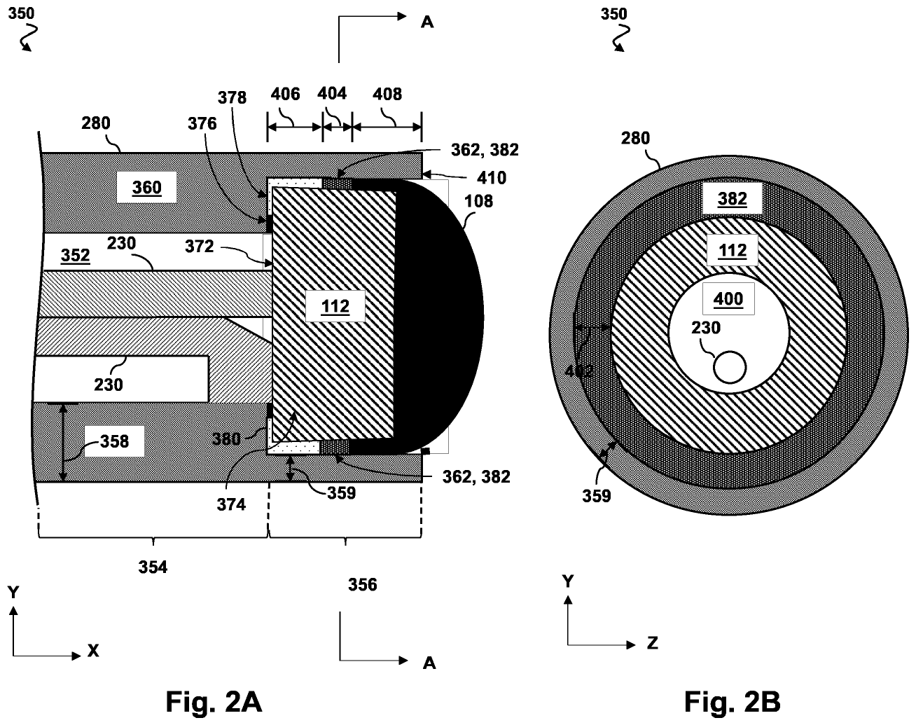

Turning now to FIGS. 2A-2B, 3A-3B, 4A-4B, and 5, diagrammatic cross-sectional views of example sensor assemblies, which may be included in the intravascular device 102 of FIG. 1, are shown. More specifically, FIGS. 2A-2B, 3A-3B, 4A-4B, and 5 illustrate exemplary sensor assemblies that include a sensing component 112 with an exposed conductive surface (e.g., a surface left uncoated by an insulating material) positioned within a housing 280 that includes a non-conductive material. As illustrated in FIG. 1, such sensor assemblies may be included in a distal portion of the intravascular device 102, as indicated by the positions of the sensing component 112 and the housing 280.

FIG. 2A illustrates a diagrammatic cross-sectional side view of a sensor assembly 350. As illustrated, the sensor assembly 350 includes the sensing component 112 positioned within the housing 280. As further illustrated, a portion (e.g., a distal portion) of the multi-filar conductor bundle 230 extends through the housing 280 and is electrically coupled (e.g., in electrical communication) with the sensing component 112. In this regard, at least a portion of the multi-filar conductor bundle 230 may extend through a hole or cutout within the sensing component 112 in some embodiments. Cut plane A-A shows a cross-sectional plane through the distal portion of the sensor assembly 350, which illustrates the hole and is shown in greater detail in FIG. 2B.

While the sensing component 112 and the housing 280 are illustrated as having a particular configuration (e.g., shape, structural arrangement, and/or the like), embodiments are not limited thereto. In this regard, while the sensing component 112 is illustrated and described as having a trapezoidal profile in the side view illustrated in FIG. 2A and a circular profile in the cut plane A-A of FIG. 2B, the sensing component 112 may have any suitable shape or dimensions. For instance, the sensing component 112 may include one or more planar portions. Moreover, the sensing component 112 may have a constant height with respect to the illustrated y-axis (e.g., a rectangular profile within the side view of FIG. 2A), or the sensing component 112 may be arranged such that the height of the sensing component 112 increases moving proximally to distally. Further, while the housing 280 is illustrated and described as being cylindrical, the housing 280 may have any suitable shape and/or dimensions. For instance, the housing 280 may include one or more planar portions. Moreover, while the housing 280 is illustrated in FIG. 2A with a relatively constant (e.g., uniform) outer dimension (e.g., outer diameter) with respect to the y-axis, this dimension of the housing 280 may vary with respect to the longitudinal axis of the housing 280 (e.g., along the x-axis). Accordingly, references to circular, cylindrical, annular configurations and/or dimensions are intended to be exemplary and not limiting.

The sensing component 112 is illustrated as being positioned within the housing 280 such that a front surface 370 of the sensing component 112 faces distally and a rear surface 372 of the sensing component faces proximally. In some embodiments, the sensing component 112 may include a transducer element, such as an ultrasound transducer element on the front surface 370 such that the transducer element faces distally and may be used by the sensing component 112 to obtain sensor data corresponding to a structure distal of the sensing component. The sensing component 112 may additionally or alternatively include a transducer element on the rear surface 372 such that the transducer faces proximally and may be used to obtain sensor data corresponding to a structure proximal of the sensing component. A transducer element may additionally or alternatively be positioned on a side surface 374 (e.g., perimeter or circumferential) of the sensing component 112 in some embodiments.

In some embodiments, one or more of the front surface 370, the rear surface 372, or the side surface 374 of the sensing component 112 may form a conductive surface, such as a metal surface, or include a conductive portion. In an exemplary embodiment, a transducer (e.g., an ultrasound transducer) may be positioned on the front surface 370 and/or the rear surface 372, and the side surface 374 may be formed from a conductive material (e.g., a metal) such that the side surface forms the conductive surface. In this way, a perimeter (e.g., a circumference) of the sensing component 112 may include the conductive surface in some embodiments. Moreover, the conductive surface may be an exposed (e.g., uncoated) surface. In this regard, the conductive surface may lack an insulative layer or coating, such as a parylene coating. In the embodiments described, the side surface 374 of the sensing component 112 is referred to as an exposed conductive surface. However, embodiments are not limited thereto. In this regard, the front surface 370) and/or the rear surface 372 may additionally or alternatively include an exposed conductive surface. To that end, the techniques described herein may be applied to the exposed conductive surface of any combination of surfaces or features of the sensing component 112.

As further shown in FIG. 2A, the housing 280 may include a hollow interior 352, as well as a proximal portion 354 and a distal portion 356. In some embodiments, a shape of the hollow interior 352 within the proximal portion 354 may vary from the shape of the hollow interior 352 within the distal portion 356. More specifically, the hollow interior 352 may be shaped to receive the sensing component 112 within the distal portion 356 and to receive the multi-filar conductor bundle 230 within the proximal portion 354. For instance, as illustrated, a thickness 358 of a wall (e.g., an inner diameter) of the housing 280 at the proximal portion 354 may be different (e.g., thicker) than a thickness 359 of the wall at the distal portion 356. In some embodiments, the thickness of the wall may remain relatively constant between the proximal portion 354 and the distal portion 356 and/or across a length of the housing 280. Further, while the housing 280 is illustrated as having a relatively constant outer profile, embodiments are not limited thereto. To that end, to receive the sensing component 112 at the distal portion 356, the outer profile may have a different height with respect to the illustrated y-axis (e.g., a lateral axis of the housing 280) at the distal portion 356 than the proximal portion 354 in some embodiments.

In some embodiments, the housing 280 may include a conductive material 360 (shown with a first fill pattern), such as a metal, as well as a non-conductive material 362 (shown with a second fill pattern), such as a ceramic and/or a polymer. In particular, the hollow interior 352 may be defined by a continuous surface (e.g., an integrally formed surface) that includes a first portion including the conductive material 360 (e.g., a conductive portion) and a different, second portion including the non-conductive material 362 (e.g., a non-conductive portion). As described in greater detail below, the housing 280 may be formed via a process, such as an additive process, such that the first and second portions are integrally formed in a unitary component. Further, in some embodiments, the non-conductive material may be included in one or more projections 382 that project (e.g., extend) radially inward from the housing 280.

As illustrated in FIG. 2B, the projection 382 of non-conductive material 362 surrounds the perimeter (e.g., circumference) of the side surface 374 of the sensing component 112. More specifically, the projection 382 shown in FIGS. 2A-2B is an annular projection that contacts a circumference of the sensing component 112. As further shown in FIG. 2A, the housing 280 contacts the sensing component 112 only at the projection 382. That is, for example, the portion of the sensing component 112 that is not in contact with the projection 382 is spaced from the housing 280. Moreover, as described above, the side surface 374, which includes the circumference of the sensing component 112, may be formed from a conductive material (e.g., forming the exposed conductive surface). Accordingly, the non-conductive material 362 of the projection 382 may contact and electrically isolate the exposed conductive surface (e.g., the side surface 347) of the sensing component 112 from the conductive material 360 of the housing 280. Thus, the projection 382 may prevent a short (e.g., an electrical short) between the sensing component 112 and the housing 280. In this way, the projection 382 may prevent an electrical failure of the sensing component 112.

The projection 382 may further secure the sensing component 112 within the housing 280. In this regard, the projection 382 may be configured to control the position of the sensing component 112 within the housing 280. For instance, a thickness 402, a length 404, a placement (e.g., a distance 406 from a proximal end of the distal portion 356 and/or a distance 408 from a distal end of the housing 280), and/or the like of the projection 382 may be controlled to arrange the sensing component 112 within the housing 280 at a particular position. For instance, with a sensing component 112 having a decreasing height (e.g., diameter) from the rear surface 372 to the front surface 370, increasing the thickness 402 of the projection 382 may cause the sensing component 112 to contact and/or engage with the projection 382 at a portion of the sensing component with a relatively greater height (e.g., closer to the rear surface 372) during assembly of the sensor assembly 350. On the other hand, decreasing the thickness of the projection 382 may cause the sensing component 112 to contact and/or engage with the projection 382 at a portion of the sensing component with a relatively smaller height (e.g., farther from the rear surface 372) during assembly of the sensor assembly 350. Accordingly, this relationship between the height of the sensing component 112 and the thickness 402 of the projection may control where the sensing component 112 sits within the housing 280 along the longitudinal axis of the housing 280 (e.g., along the illustrated x-axis). Similarly, by adjusting the length 404, the distance 406, and/or the distance 408, the position of the sensing component 112 along the longitudinal axis of the housing 280 may be adjusted.

Moreover, while the thickness 402 is illustrated as relatively uniform in FIGS. 2A-2B, in some embodiments, the thickness 402 may vary across the inner perimeter of the housing 280. In this regard, the distribution of thicknesses across the inner perimeter of the housing 280 may control the positioning of the sensing component 112 along the plane defined by the illustrated y-axis and z-axis. Further, while the length 404 and the placement (e.g., the distances 406 and 408) are illustrated as relatively uniform in FIG. 2A, the length 404 and/or the placement of the projection 382 may vary across the inner perimeter of the housing 280, which may further control the position of the sensing component 112 within the housing 280. In any case, the projection 382 may affect the positioning of the sensing component 112 within the housing 280 and may secure the sensing component 112 within a particular position, which may ensure that the positioning of the sensing component 112 is not disturbed by the addition of other components to the sensor assembly 350, such as the distal tip 108. In this regard, the arrangement of the projection 382 may ensure the exposed conductive surface of the sensing component 112 remains spaced from the conductive material 360 of the housing during assembly of the sensor assembly 350 and within the sensor assembly 350, once manufactured.

FIG. 2B further illustrates the hole 400 (e.g., cutout), which may receive one or more conductive filars of the multi-filar conductor bundle 230. In the illustrated embodiment, one filar (e.g., conductive member) of the multi-filar conductor bundle 230) are shown extending through the hole 400. In some embodiments, a filar extending through the hole 400 may electrically couple to an element, such as a transducer, on the front surface 370) of the sensing component 112. However, embodiments are not limited thereto. In this regard, one or more conductive filars of the multi-filar conductor 230 may terminate at (e.g., electrically couple to) the rear surface 372 of the sensing component 112. For instance, in some embodiments, the sensing component 112 may lack the hole 400. Further, in some embodiments, a subset of the filars of the multi-filar conductor bundle 230 may extend through the hole 400 and/or electrically couple to an element at the front surface 370, while a different subset of the filars may not extend through the hole 400. The different subset may electrically couple to an element at the rear surface 372, for example. In particular, a first filar may extend through the hole 400, while a different, second filar may terminate at the rear surface 372. Further, in some embodiments, regardless of whether the multi-filar conductor bundle 230 is physically coupled or directly electrically coupled to (e.g., soldered to, bonded to, and/or the like) the exposed conductive surface (e.g., the side surface 374), the multi-filar conductor bundle may be capable of electrically coupling to the side surface 374. For instance, if the side surface 374 contacts the conductive material 360 of the housing 280, the low resistance (e.g., the conductive nature) of the side surface 374 and/or the housing 280 may cause current to flow from the multi-filar conductor bundle 230 through the side surface 374 and the housing 280, thereby causing the electrical short described herein.

With reference to FIGS. 2A-2B, in some embodiments, to assemble the sensor assembly 350, the multi-filar conductor bundle 230 may be coupled (e.g., physically coupled) to the sensing component 112 to form a sub-assembly. The sub-assembly may be positioned within the housing 280 (e.g., within the hollow interior 352 of the housing 280) by sliding the proximal portion 354 over the multi-filar conductor bundle 230. In some embodiments, the housing 280) may include an adhesive 376 within the proximal portion 354 to secure the sensing component 112 to the housing 280. Further, as illustrated, in some embodiments, the adhesive may be positioned on a distal wall 378 within the proximal portion 354. In some embodiments, the housing 280 may additionally or alternatively include an acoustic backing 380. The acoustic backing 380 may be the same material or different material than the adhesive 376. Further, as illustrated, the acoustic backing 380) may contact the distal wall 378 and/or a side wall 379 within the distal portion 356. In particular, the acoustic backing 380 may be applied such that the acoustic backing 380 contacts the rear surface 372 as well as a portion of the side surface 374 when the sensor assembly 350 is assembled. In other embodiments, application of the acoustic backing 380 may be controlled such that the acoustic backing 380 contacts only the rear surface 372 when the sensor assembly 350 is assembled.

As further illustrated in FIGS. 2A-2B, because the projection 382 completely surrounds the perimeter of the sensing component 112, the acoustic backing 380 and/or the adhesive 376 may not be introduced into the housing 280 after the sub-assembly of the sensing component 112 and multi-filar conductor bundle 230 is positioned within the housing 280. Instead, the acoustic backing 380 and/or the adhesive 376 may be introduced to the housing 280 before introduction of the sub-assembly. In this regard, the arrangement of the projection 382 may prevent introduction of a material (e.g., an adhesive and/or an acoustic backing material) distal of the projection 382 once the projection 382 is in contact with the sensing component 112.

In some embodiments, the sensor assembly 350 may include a distal tip 108 (e.g., an atraumatic tip). As illustrated in FIG. 1, by positioning the sensor assembly 350 at the distal end of the intravascular device 102, the distal tip 108 may form the distal tip of the intravascular device 102. In some embodiments, the distal tip 108 may be formed from a material that provides acoustic matching with the sensing component 112. That is, for example, the distal tip 108 may serve as an acoustic matching layer with the sensing component 112. As disclosed in U.S. Pat. No. 10,973,419, titled “INTRAVASCULAR PRESSURE DEVICES INCORPORATING SENSORS MANUFACTURED USING DEEP REACTIVE ION ETCHING,” which is hereby incorporated by reference in its entirety, the distal tip 108 may be formed from a highly elastic material, such as a soft silicone elastomer, that transmits pressure applied at the distal end of the intravascular device 102 to the sensing component 112 (e.g., to a surface of the sensing component 112 that includes an ultrasound transducer). An example of such a highly elastic material is a low durometer silicone elastomer, such as MED-4905, MED 4930, and/or the like from NuSil Technology LLC of Carpinteria, Calif.

As illustrated, the distal tip 108 may cover the front surface 370 of the sensing component 112 and may cover a portion of the side surface 374 of the sensing component 112. In some embodiments, the distal tip 108 may cover the front surface 370 of the sensing component 112 but may not extend to the side surface 374 of the sensing component 112. Further, in some embodiments, the distal tip 108 may cover a distal end 410 of the housing 280. Moreover, while the distal tip 108 is illustrated as having a domed shape, embodiments are not limited thereto. In this regard, the distal tip 108 may include a flattened profile or any suitable shape. Additionally or alternatively the distal tip 108 may be formed from one or more layers of materials. The layers may include different materials and/or different configurations (e.g., shape and/or profile, thickness, and/or the like).

In some embodiments, the housing 280 may be formed via an additive process, as described above. In particular, the housing 280 may be constructed from a manufacturing method, such as three-dimensional (3D) printing, photolithography, electrodeposition, and/or other suitable processes (e.g., micro-electromechanical system (MEMs) and/or semiconductor manufacturing processes), that involves forming the housing 280 from a plurality of layers. For instance, the plurality of layers may be formed atop one another according to any combination of the above manufacturing techniques such that the layers define a continuous (e.g., integral) surface of the housing 280. In this way, the housing 280 may be formed as a unitary component. Further, the continuous surface of the housing 280 may be formed via the additive process to include one or more projections, such as projection 382. Moreover, the plurality of layers may include a first layer that includes the conductive material 360 and a second layer that includes the non-conductive material 362 such that the housing 280 includes a conductive portion and a non-conductive portion, respectively. Further, in some embodiments, individual layers of the plurality of layers may be formed with different materials, thicknesses, height, lengths, shapes, and/or like as one another. In this way, characteristics of the housing 280 may be tuned via selection of the layers used to form the housing.

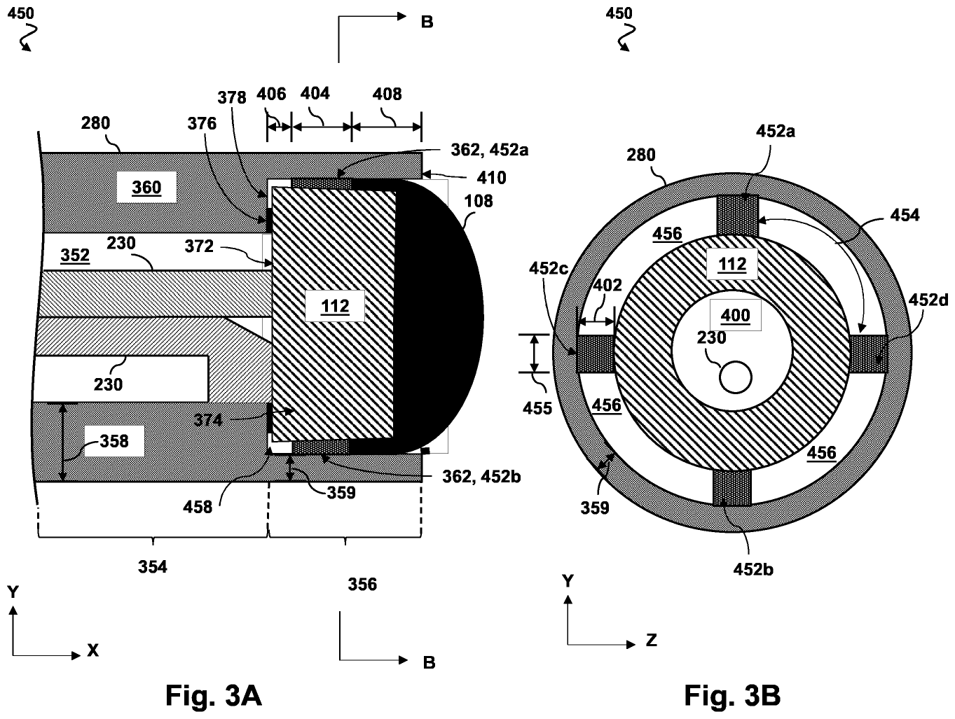

In some embodiments, the housing 280 may include multiple projections of non-conductive material 362, as illustrated in FIGS. 3A-3B. FIG. 3A illustrates a diagrammatic cross-sectional side view of a sensor assembly 450. FIG. 3A includes a cut plane B-B, which shows a cross-sectional plane through the distal portion of the sensor assembly 450 and is illustrated in FIG. 3B. As illustrated, the components illustrated in the sensor assembly 450 are substantially similar to the corresponding components illustrated in the sensor assembly 350 of FIGS. 2A-2B. In the illustrated sensor assembly 450, however, the housing 280 includes a plurality of projections that are formed from the non-conductive material 362 and are spaced from one another. More specifically, the sensor assembly 450 includes multiple linear projections 452a-d that are positioned in the distal portion of the housing 280, spaced from one another, and arranged to contact the sensing component 112 within the sensor assembly 450. For instance, within the side view of FIG. 3A, the linear projections 452a-b are shown, and in the cut plane illustrated in FIG. 3B, the linear projections 452a-b, as well as the spacing 454 between the linear projections 452a-b are shown. As described herein, the term “linear projection” refers to a projection that is arranged to contact the sensing component 112 over a first portion of the sensing component 112, such as a first line or area of the sensing component 112. The linear projections 452 may be implemented in any suitable shape, which may or may not include planar surfaces. For instance, the linear projections 452 may be implemented with a relatively cylindrical shape, a rectangular prism shape, and/or the like.

The spacing 454 of FIG. 3B illustrates that multiple projections, such as the illustrated linear projections 452a-d) may be spaced from one another along a perimeter (e.g., a circumference) of the housing 280. While the spacing 454 is illustrated as relatively constant between each of the linear projections 452a-d, the spacing 454 may vary between pairs of projections in some embodiments. For instance, in some embodiments, the spacing 454 between particular linear projections 452 may be controlled to affect positioning of the sensing component 112 within the housing 280, as described above with respect to control of other characteristics (e.g., the thickness 402, length 404, distance 406 from a proximal end of the distal portion 356 and/or a distance 408 from a distal end of the housing 28) of projections of the housing 280. To that end, the distance between two projections (e.g., the spacing 454), the height 455 of the projections with respect to the y-axis, the thickness 402 of the projections and/or the like may be adjusted and may vary from projection to projection. Moreover, while the sensor assembly 450 is illustrated as including four linear projections 452a-d, the sensor assembly 450 may include any suitable number of linear projections (e.g., one, two, three, five, eight, and/or the like).

As further illustrated by FIG. 3B, the arrangement (e.g., the spacing 454, height 455, thickness 402, quantity, and/or the like) of the linear projections 452a-d may define one or more gaps 456. In particular, the arrangement of the linear projections 452 of the sensor assembly 450 may determine the quantity, size, and positioning of the gaps 456 within the housing 280. Again, while the sensor assembly 450 is illustrated as having four projections (e.g., linear projections 452a-d) defining four gaps 456, a sensor assembly may include any number of projections defining any suitable number of gaps. In some embodiments, for example, a sensor assembly may include a single projection defining a single gap 456. For instance, the single projection may surround a portion of the sensing component 112 such that opposite ends of the projection are spaced from one another by the gap 456.

Further, the gaps 456 may allow air or another material to flow and/or otherwise be introduced into the housing 280 (e.g., via the gaps 456) after the sub-assembly of the sensing component 112 and multi-filar conductor bundle 230 is positioned within the housing 280. As such, in some embodiments, the gaps 456 may facilitate air and/or another suitable gas occupying the space 458 within the distal portion 356 of hollow interior 352 that is distal of the sensing component 112. For instance, the space 458 may correspond to the volume filled with acoustic backing 380 (e.g., an acoustic backing material) and/or the adhesive 376 in the sensor assembly 350 of FIG. 2A. In such embodiments, air may be used as an acoustic backing for the sensing component 112. For example, ultrasound energy does not propagate well through air such that air can be used for attenuation of ultrasound energy in undesired directions (e.g., backwards, sideways). As further illustrated, the air or another acoustic backing material may be sealed within the sensor assembly 450 by the distal tip 108.

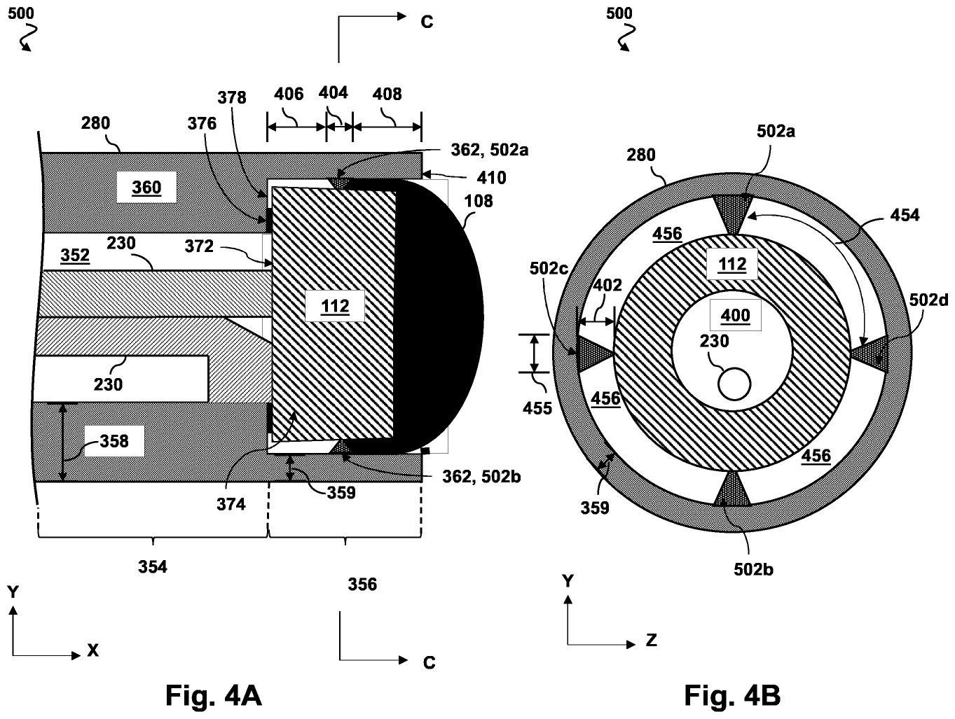

In some embodiments, the housing 280) may include one or more projections with an alternative shape, such as a conical or pyramid shape, as illustrated in FIGS. 4A-4B. FIG. 4A illustrates a diagrammatic cross-sectional side view of a sensor assembly 500. FIG. 4A includes a cut plane C-C, which shows a cross-sectional plane through the distal portion of the sensor assembly 500 and is illustrated in FIG. 4B. As illustrated, the components illustrated in the sensor assembly 500 are substantially similar to the corresponding components illustrated in the sensor assembly 350 of FIGS. 2A-2B and/or the sensor assembly 450 of FIGS. 3A-3B.

Similar to the sensor assembly 450, the illustrated sensor assembly 500 includes a plurality of projections within the housing 280 that are formed from the non-conductive material 362 and are spaced from one another. More specifically, the sensor assembly 450) includes multiple point-contact projections 502a-d that are positioned in the distal portion of the housing 280, spaced from one another, and arranged to contact the sensing component 112 within the sensor assembly 500. For instance, within the side view of FIG. 4A, the point-contact projections 502a-b are shown, and in the cut plane illustrated in FIG. 4B, the point-contact projections 502a-b, as well as the spacing 454 between the point-contact projections 502a-b are shown. In comparison with the sensor assembly 450, the point-contact projections 502 illustrated in the sensor assembly 500 are arranged to contact the sensing component 112 over a smaller surface area (e.g., over a spot or point on the sensing component 112) than the linear projections 452. In this regard, the term “point-contact projection” refers to a projection that is arranged to contact the sensing component 112 over a first portion of the sensing component 112, such as a spot or point (e.g., a relatively small area) of the sensing component 112. While the point-contact projections 502 are illustrated as having a conical or pyramid shape, the point-contact projections 502 may be implemented in any suitable shape. For instance, the point-contact projections 502 may be implemented with a relatively spherical or domed shape, a rectangular shape, and/or the like. Moreover, while the sensor assembly 500 is illustrated as including four point-contact projections 502a-d, the sensor assembly 500 may include any suitable number of point-contact projections (e.g., one, two, three, five, eight, and/or the like).

In comparison with the projection 382 of FIGS. 2A-2B and in further comparison with the linear projections 452 of FIGS. 3A-3B, the point-contact projections 502 may be formed with less material (e.g., non-conductive material 362). Accordingly, the sensor assembly 500 may be produced with relatively fewer resources. Further, the point-contact projections 502 may have a relatively smaller profile than the linear projections 452. Accordingly, the spacing 454 between the point-contact projections 502 may be more readily increased, thereby increasing the size of gaps 456.

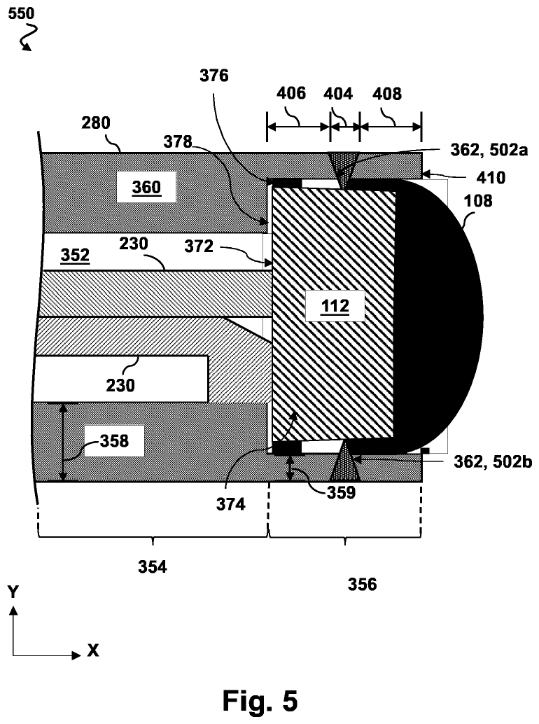

In some embodiments, a non-conductive portion of the housing 280 (e.g., a portion formed from the non-conductive material 362) may extend through a portion of the wall 358 of the housing 280, as illustrated in FIG. 5. FIG. 5 illustrates a diagrammatic cross-sectional side view of a sensor assembly 550. As illustrated, the components illustrated in the sensor assembly 550 are substantially similar to the corresponding components illustrated in the sensor assembly 350 of FIGS. 2A-2B, the sensor assembly 450 of FIGS. 3A-3B, and/or the sensor assembly 500 of FIGS. 4A-4B.

While the non-conductive portions (e.g., projections) of the housing 280 shown in the sensor assembly 350 of FIGS. 2A-2B, the sensor assembly 450 of FIGS. 3A-3B, and the sensor assembly 500 of FIGS. 4A-4B are shown as being spaced from an outer profile (e.g., a circumference) of the housing 280), the projections (e.g., the point-contact projections 502a-b) of the sensor assembly 550 are illustrated as extending to the outer profile of the housing 280. In this regard, the projections shown in FIG. 5 extend through the thickness 359 of the wall at the distal portion 356. The illustrated embodiment is intended to be exemplary and not limiting. In this regard, in some embodiments, a non-conductive portion of the housing may be formed such that the non-conductive portion extends through any suitable portion of the thickness 359 and/or the thickness 358.

The sensor assembly 550 further illustrates that the adhesive 376 may be positioned in the housing 280 such that the adhesive 376 contacts the side surface 374 of the sensing component. In this regard, the adhesive 376 may be positioned at any suitable position or combination of positions within the housing 280. Moreover, while not illustrated, the hollow interior 352 may be filled or partially filled with a material within the proximal portion 354. For instance, the multi-filar conductor bundle 230 may be at least partially surrounded by air, an adhesive, an acoustic backing material, and/or the like within the proximal portion 354.

Embodiments described herein are intended to be exemplary and not limiting. In this regard, one or more of the illustrated components in a sensor assembly may be omitted, additional components may be added, and two components illustrated as separate may represent a single component. Moreover, while the non-conductive portions (e.g., projections) of the housing 280 shown in the sensor assembly 350 of FIGS. 2A-2B, the sensor assembly 450 of FIGS. 3A-3B, and the sensor assembly 500 of FIGS. 4A-4B, and the sensor assembly 550 of FIG. 5 are shown as projecting into the hollow interior 352, in some embodiments, the non-conductive portion of the housing 280 may be formed such that the non-conductive portion of the housing 280 is flush with the conductive portion. That is, for example, embodiments are not limited to non-conductive portions of the housing 280 being projections. In such embodiments, the sensing component 112 may be shaped to contact the non-conductive portion and remain spaced from the conductive portion, for example. Further, while non-conductive portions of the housing 280 are described herein as being spaced from one another along the perimeter of the housing 280 with respect to the plane formed by the y-axis and the z-axis, non-conductive portions may additionally or alternatively be spaced from one another along the x-axis (e.g., with respect to the longitudinal axis of the housing 280). Moreover, in some embodiments, the non-conductive portion may extend across the distal portion 356 of the housing 280. In particular, the surface of the housing 280 within the hollow interior 352 of the distal portion 356 may be entirely defined by the non-conductive material 362. For instance, the non-conductive material 362 may be integrally formed with the housing 280 to form the continuous surface of the housing 280, as described herein, or the non-conductive material 362 (e.g., an insulating material) may be coated on a portion of the housing 280. In some embodiments, for example, a portion of the housing 280 may include a parylene coating.

A person of ordinary skill in the art will recognize that the present disclosure advantageously provides a sensor assembly that eliminates an insulating layer on a sensing component and maintains electrical isolation between the sensing component and a housing for the sensing component. In particular, the housing includes a non-conductive portion that is arranged to contact the sensing component and electrically isolate the sensing component from a conductive material of the housing. The logical operations making up the embodiments of the technology described herein are referred to variously as operations, steps, objects, elements, components, regions, etc. Furthermore, it should be understood that these may occur in any order, unless explicitly claimed otherwise or a specific order is inherently necessitated by the claim language.

It should further be understood that the described technology may be employed in a variety of different applications, including but not limited to human medicine, veterinary medicine, education and inspection. All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the intraluminal imaging system. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intraluminal sensing device, comprising:
a flexible elongate member comprising a distal portion and a proximal portion and configured to be positioned within a body lumen of a patient;
a housing positioned at the distal portion of the flexible elongate member, wherein the housing comprises a conductive material and a non-conductive material; and
a sensor comprising an exposed conductive surface and configured to obtain physiological data while positioned within the body lumen, wherein the sensor is positioned within the housing such that the exposed conductive surface contacts the non-conductive material of the housing and is electrically isolated from the conductive material.

2. The intraluminal sensing device of claim 1, wherein the exposed conductive surface comprises a perimeter of the sensor.

3. The intraluminal sensing device of claim 1, wherein the non-conductive material comprises a ceramic material.

4. The intraluminal sensing device of claim 1, wherein the non-conductive material comprises a polymer.

5. The intraluminal sensing device of claim 1, wherein the housing comprises a hollow interior defined by a continuous surface, wherein a first portion of the continuous surface comprises the conductive material and a different, second portion of the continuous surface comprises the non-conductive material.

6. The intraluminal sensing device of claim 5, wherein the housing comprises a plurality of layers formed atop one another such that the plurality of layers defines the continuous surface, wherein a first layer of the plurality of layers comprises the conductive material and is disposed within the first portion of the continuous surface and a different, second layer of the plurality of layers comprises the non-conductive material and is disposed within the second portion of the continuous surface.

7. The intraluminal sensing device of claim 5, wherein the sensor is positioned within the hollow interior such that the exposed conductive surface contacts the second portion of the continuous surface and is spaced from the first portion of the continuous surface.

8. The intraluminal sensing device of claim 1, wherein the housing comprises a first projection extending within a hollow interior of the housing, wherein the first projection comprises the non-conductive material and is in contact with the exposed conductive surface.

9. The intraluminal sensing device of claim 8, wherein the first projection surrounds a perimeter of the exposed conductive surface.

10. The intraluminal sensing device of claim 8, wherein:
the housing comprises a different, second projection extending within the hollow interior, wherein the second projection comprises the non-conductive material and is in contact with the exposed conductive surface;
the first projection is arranged to contact the exposed conductive surface over a first portion of the exposed conductive surface; and
the second projection is arranged to contact the exposed conductive surface over a different, second portion of the exposed conductive surface, wherein the second portion is spaced from the first portion.

11. The intraluminal sensing device of claim 1, further comprising an air gap positioned between the sensor and the housing, wherein the air gap comprises an acoustic backing layer.

12. The intraluminal sensing device of claim 1, wherein the sensor comprises a flow sensor.

13. The intraluminal sensing device of claim 1, further comprising a wire assembly electrically coupled to the exposed conductive surface.

14. The intraluminal sensing device of claim 1, further comprising an adhesive positioned between the sensor and the housing.

15. The intraluminal sensing device of claim 1, wherein the housing comprises a coating of the non-conductive material.

16. An intraluminal flow-sensing device, comprising:
a guidewire comprising a distal portion and a proximal portion and configured to be positioned within a blood vessel of a patient;
a housing positioned at the distal portion of the guidewire and comprising a hollow interior defined by a continuous surface, wherein a first portion of the continuous surface comprises a conductive material and a different, second portion of the continuous surface comprises a non-conductive material; and
a flow sensor comprising an exposed conductive surface and configured to obtain intravascular flow data while positioned within the blood vessel, wherein the flow sensor is positioned within the hollow interior of the housing such that the exposed conductive surface contacts the second portion and is electrically isolated from the first portion.
